(19) United States
(12) Patent Application Publication
(10) Pub. No.: US 2018/0261307 A1
(43) Pub. Date: Sep. 13, 2018

(54) SECURE MONITORING OF PRIVATE ENCOUNTERS

(71) Applicant: SPXTRM HEALTH INC., Toronto (CA)

(72) Inventors: John M. COUSE, Toronto (CA); Jesse SLADE SHANTZ, Toronto (CA)

(21) Appl. No.: 15/891,840

(22) Filed: Feb. 8, 2018

Related U.S. Application Data
(60) Provisional application No. 62/457,521, filed on Feb. 10, 2017.

(30) Foreign Application Priority Data
Feb. 10, 2017 (CA) 2957567

(51) Int. Cl. G16H 10/60; H04L 29/06; G16H 80/00; H04N 7/18

(52) U.S. Cl. CPC G16H 10/60; H04N 7/18; G16H 80/00; H04L 63/0428

(57) ABSTRACT

A method and system for the secure recording, uploading and management of data, audio and video relating to person-to-person encounters to protect both participants from improper behavior in a closed setting, by either party, and to support the rules of digital of evidence in the case of a potential investigation all the while protecting the privacy of the participating parties.

### TECHNICAL FIELD

One or more aspects disclosed herein relate to the secure capture, uploading, and management of data, audio and video information relating to private encounters in various person-to-person clinical, commercial or other service delivery settings.

### BACKGROUND

In terms of the Patient/Provider relationship, it is one of the most sacred interactions that a person has in their life and where there is a high expectation of professionalism, trust and privacy including zero tolerance for sexual abuse by either party. The terms of this relationship are enshrined in the Hippocratic Oath. Traditionally, in this intimate setting the professional commitment of the provider was all that was necessary to protect the patient against violations of the boundary between patient and provider. Now, it has become expected, especially if members of the opposite sex or sexual orientation were present, that there would be a 3^{rd} person present in the room in the form of a patient-supplied guardian or an employee of a medical professional or institution where the exam is taking place. Regardless of your gender, a provider should have a chaperone consistently available for intimate exams, according to the American Medical Association Code of Medical Ethics.

With the continued downward pressure on reimbursement levels available from payer's (insurance companies, government programs, corporate self-funded programs, individuals etc.) or from competition that is present in some markets, the requirement for a chaperone in the room has started to have an effect on the margins for providers and institutions.

Unfortunately, without a chaperone present in the examination room, there is the possibility of misconduct by the Provider or the Patient and/or the chance that one party has misunderstood something that was either said or done. Real or false accusations of misconduct are hard to prove at the regulatory body, civil or judicial level as it will come down to one party's word against another. The consequences of such an investigation are devastating for either party as it will entail endless stressful meetings, depositions, and ongoing investigations. It can result in missing work, receiving negative public media exposure and the Provider receiving a suspension or loss of their practicing license. In summary, if there has been any possibility of misconduct by either party, they should be disciplined or exonerated through the regulatory body and/or through local civil and judicial means immediately, with as little disruption to the other party's life. Currently, if a sexual abuse victim does come forward they must endure a ruthless cross-examination in front of the alleged abuser and relive the incident

Individuals are increasing their public profile through the sharing of personal images as well using video telehealth services. It is forecasted that by the year 2020, 80% of all healthcare interactions will take place through a video interface of some type or other. Providers are also starting to use mobile phones to record and share medical images for consultation with known specialists on a one-to-one basis as well as sharing anonymous images with a larger general medical population.

Current video based Patient/Provider solutions, in a physical healthcare setting are currently used either to record the physician's instructions provided at the end of the examination session for later non-synchronous review by the Patient and their personal support network (e.g. family and friends) or for the sharing of personal images of a patient's condition to a physician or for the physician to share image with other physicians for consults.

In some cases, these types of systems are designed for public viewing with a supplied URL and PIN number. In other cases, any identifying personal health information is removed from the images(s) before posting, usually by manual review. In either case, these system's inherent limitations allow for the images to be eventually be propagated and shared/posted online without the participant's consent.

The Patient/Provider (the Patient, the Provider) encounters referenced above may also refer to any encounter where there is the high expectation of privacy and trust. This may include any type of encounter that takes place in a medical, health, fitness, spiritual, sports, commercial or education setting and where Providers could be classified either as physicians, nurses, therapists, medical technicians, massage therapists, coaches, educators, spiritual counselors, sales agents, consultants or religious personnel etc.

### SUMMARY

The best solution to address the growing issue improper behavior between two parties is to provide an effective deterrent in the examination or meeting room so that a sexual abuse incident or any other type of misconduct does not occur in the first place. For example, in the law enforcement field, there has been an expanding use of body cameras with some departments using body-worn cameras and microphones for enforcement and conviction purposes while other departments are using it as a deterrent to reduce improper behavior by both the police officers and the public. Current studies suggest that body cams are more effective as a deterrent than they are as an enforcement and conviction tool. Statistics have been published where the use of deadly force has dropped by 58% and citizen's complaints has dropped by 88% through the use of video recorded encounters

What is proposed is a smart video and audio recording device(s) (the Device), that is physically secured in a given space, or on a person, and preferably with a forward-facing display screen, that is placed in the encounter session location, for the benefit of both the person receiving the service and the person providing the service or advice. The Device may be all inclusive in a single package, such as mobile tablet Device or a customized Device that includes lenses, charged coupled device's (CCD's), a vision processing unit (VPU), display screen, micro controllers, memory, communication components, and software or it may include external cameras with wide angle or 360 degree lenses and special sensitive audio input/output devices connected to another computing and communicating device using broadband or cellular services. The Device may also be in the form of a local networked image recording Device, in the case of a multi-examination room, that connects to a local secure server and handles all of the store, forward and upload functions to the cloud based service. The Device may also be in the form of a body worn cameras in various different embodiments situated on different parts of the Provider's body. The examples and figures are illustrative rather than limiting.

The recording Device will have specific downloaded software to control various functions or operate through an on-line browser interface to operate in a software-as-a-service model. These software functions include the ability to authenticate the Patient through touch screen prompts, digital signatures and/or voice and face recognition. It will also use image recognition techniques on the Patient's health card and match it through API's to various government and commercial payer's 3^{rd} party systems. The authentication and enrollment can be secured by the front desk administrator, the attending nurse or by the Patient themselves, through voice chat bots and/or touch screen commands. It can take place on a remote basis ahead of the appointment, at the check-in desk or in the actual examination room. During enrollment, the system will request a case number from the central server and pre-populate some of the required fields of the metadata so that the Provider can begin the recorded session with a specific Patient without delay when they become available. The request of a case number will prompt various functions at the central server such as sending a text or email to the Patient or guardian's personal account, updating the Provider's on-line accessible account and/or the Provider's electronic medical record system (EMR).

The Device will have the ability to run pre-exam tests and questionnaires with the Patient using screen prompts, a digital avatar or a chatbot, the ability to be self-aware that there is a potential recording event to take place, that the lighting conditions are satisfactory to capture the desired event, and that the individuals are actually in frame with appropriate audio prompts to support the required outcome using computer vision (CV) technology. The software will also block the use of the Device's onboard camera and microphone from being inadvertently (or intentionally triggered by the Provider to illegally record the disrobing or robing of the Patient) operating and capturing unauthored video. To start an actual recording session, the system may be triggered by screen prompts, facial recognition, fingerprint recognition, or various RFID techniques. Features described above are designed to protect the privacy of the individuals and reduce the number of erroneous recordings and transmissions as well as to facilitate a digital contract and consent between the Patient and Provider that covers the digital rights to the data, audio and video obtained.

In another embodiment, the Patient's wearable fitness and health data (e.g. Fitbit) will be uploaded into the recording Device using Wi-Fi, low energy Bluetooth (BLE), LoRA, light, sound, or other transmission technology and will be available for review by the Provider and the Patient. Such data may be displayed/superimposed over the Device's display screen while the Device is still recording the pre-approved session.

As well as the Device being able to recognize faces it will provide biometric feedback on each participating party's vital sighs (e.g. HR, HRV, respiration, blood pressure etc.) through image based photoplethysmograthy (PPG). It will also provide for an analysis of the participant's stress levels, emotional levels and look for vocal biomarkers (e.g. coronary artery disease (CAD), Parkinson's disease, postpartum depression etc.) using both image and voice AI recognition. Another feature of the Device and/or server software combination is its ability to combined audio, image and sensory input to determine high risk events that would either trigger a real-time alarm and/or mark the session as problematic to warrant further investigation.

In another embodiment, the Device, either independently or with assistance from the cloud based server, will also support the use of an imbedded symptom checker that can be driven by variables that are entered on the Device's touch screen, through voice commands or a chatbot, collected from the Patient's wearable devices or derived through AI analysis of the voice and images being collected. This may also include a real-time language translator to assist the Patient/Provider interaction. Even though additional data and apps may be displayed/overlaid on the screen, the pre-approved recording session will still continue unless the Patient and the Provider agree to a pause in the recording.

The Device, during downtime between sessions, can also display sponsored and/or educational content that is provided on an anonymous or a session contextual aware basis based on the profile of the Provider, the type of practice that they provide, the nature of the visit or the specifics of the Patient. Such sponsored content could be targeted at the Provider, the Patient or both and such fees collected by the operator of the service could be used to assist in defraying the costs of providing the service.

The Device software will add a watermark to each frame that represents the unique case number that is retrieved from the connected cloud based server as well as the sequential serial number that is obtained from the Device for each sequential session recorded. Each frame will also be hashed so that the final uploaded image file/object and stored image file/object can be checked for non-authorized editing and manipulation in the case of an investigation to support the rules of digital evidence. Missing session serial numbers is also a good way to determine if a Provider has tried to suppress the upload of a specific recorded session.

All of the external sensory data, the software session data and the Device and environmental data, such as IP address, system date and time, MAC address, IEMI number (in the case cellular communications), etc., will be part of a metadata record that will be encrypted and transmitted separately from the video and audio files/objects. The linkage between the two files/objects could be a key derived from the session's case and serial number combination or other means. At the onset of a new session, the cloud based server will provide the case number to the Device. Metadata will always have transmission priority over the actual image file/object transmission in the case of bandwidth issues, followed by the audio file/object and then the video file/object.

In another embodiment, the Patient may not be comfortable about having their image, regardless of the proposed encryption and security levels, stored in a remote location. To address these issues the image can be altered either in real-time at the Device level or it can be marked for post processing at the cloud server level. Such altering could include a redaction of faces and/or body parts or the conversion of the participants into dimensionally correct avatars in real-time. Such avatars would be able to be viewed, on the forward-facing display screen, by the participants, during the actual recording session. In the case of more advanced image recording Devices, as they become available, that include two cameras and/or the use of structured, laser, LIDAR, infrared depth sensors, spectrometers and/or polarized light, there is an opportunity to provide more accurate body and hand positioning down to the millimeter and sub millimeter level as well as analyzing the biochemical makeup of the object they are measuring. A single image sensor from the Device may also be used to complete similar measurements using forms of monocular Simultaneous Localization and Mapping (SLAM). Such capabilities would allow for the extreme accurate overlaying of internal body components such as nerves, bones, cartridge, muscle, organs etc., using augmented reality. For example, while the Patient is raising their arm up and down, the Patient can see how the underlying muscles, bones, tendons and cartilage are interacting with each other. Such an augmented view would greatly enhance the explanation, by the Provider, of what is going on underneath the skin, as in the case of a torn rotator cuff, for example.

There may also be a need to use automated or manual voice altering and/or redaction techniques to remove personal health information (PHI) from the metadata, audio or video part of the session to meet the needs of various regulatory bodies. Anonymization of the metadata, audio and video components would also be needed to support post processing big data analysis of the aggregated files/objects. The redaction/anonymizing actions, as described above, can be in real-time at the Device level, in post-production at the server level or before committing the recorded session to deep storage. Another benefit of using AI and voice recognition is that it would allow the Provider to provide an oral or text summary transcription (in various languages) of his diagnoses as well as his prescribed drug and physical treatment plan. Such audio and video would then be transcribed by the system and the Patient could receive an email, text, voice mail with the Provider's instructions and prescription(s) at the end of session for replay to their extended personal support network and presentation to the pharmacist. Such capabilities as described above can also be used to support and substantiate billing codes that can be automatically or manually created and uploaded to the payers for further adjudication and payment.

The image and audio portion of the session could be encrypted using compression technology (CODEC) such as H.264 Advanced Video Coding (AVC) or H.265 High Efficiency Coding (HEV), their successors or emerging ones such as WebM and VP9 etc., with an adjustable frames per second rate, aspect ratio, bit rate and frame rate (intraframe) depending on the rules of digital evidence for a specific jurisdiction where the recording is taking place. Such CODEC's can be based on requirements around lossless or lossy compression techniques, the available upload bandwidth, the amount of cloud storage space available, or to meet a certain overall operating expense level to make the service available at a reasonable cost to the Provider, Patient and/or sponsor of the service etc.

In another embodiment, the CODEC is not an industry standard one but a proprietary

CODEC that is known only to the Device and to the central server. This custom CODEC adds another layer of security as even if the session files/objects are intercepted, there is no available public CODEC to download that would allow viewing of files/objects. The same would be for the metadata portion of the session in that it would be compressed in a proprietary fashion only know to the Device and the server. The same is true of all data, video and audio files/objects between the Device to the server to the offline storage whether it be in transit or at rest. The objective is to render all recorded information, whether it be at rest or in-transit, into anonymous “objects” versus recognizable files/objects and known file extensions. The term file and object will be used interchangeably in this document.

In another embodiment, the audio/video files/objects (without the metadata as described above) would be further split into two separate files/objects with one containing audio and one containing video only with their own specific CODEC's. This would result in three or more different encrypted streams all following three or more different IP paths to the cloud based server(s) with a unique derived key based on the session case and serial number to link them together in post processing, if required. In another embodiment, the IP addresses across all three or more data streams (packets) could be the same or different but the Device sending and server receiving ports would be randomized using techniques such as 2 factor authentication keys that are synchronized between the Device and the server. In this case the intruder would have to have physical access to the Device or the server to try gain access to the keys. In another embodiment, the randomization algorithms/routines are replaced or augmented with a seed that is derived from the actual pixels in the recorded content.

Regardless of the encryption scheme used, the scheme cannot be dependent on the availability of the initiating recording Device as they may have been stolen or lost. To support this, the message wrapper on each of the various metadata, audio and video file/object must be a key that is already known or that can be derived by the server. This public asymmetric key could then wrap the individual files/objects that are already encrypted using symmetrical session keys allowing for double encryption of the message and its contents. With the additional encryption of the file/object through a custom CODEC, it would in turn provide for three layers of encryption as well as what is provided by HTTPS during transit.

The Device will communicate with the cloud based services through either an Ethernet or similar cable to a router, through a locally networked computer, through Wi-Fi, or through the cellular modem capabilities of the Device or other onboard RF technologies. To improve security, it is recommended that the router have a fixed IP address and that communications only take place over HTTPS or its successor. The data path that the metadata takes will be different than the one the video or audio path takes so that even if one side of the session/case is intercepted, the possibility that hackers will intercept the other parts of the session will be decreased dramatically.

As both broadband and cellular Providers, in some markets for different classes of service, have designed their networks for optimum download and not upload speeds (83% to 90% slower), large video uploads are problematic. Another issue includes the fact that as the available camera image quality improves, as measured in megapixels, it dramatically increases file/object sizes even after aggressive compression and these files/objects will take even longer to upload in the future. This can result in a day's session of videos taking substantially more time to transmit compared to the time it took to create them. As the Provider, would not leave a Device on/open or unattended when the office is closed, several features are included in the design to accommodate this issue. As mentioned previously, the request for a new case number from the server and the uploading of the metadata always has priority over other traffic, followed by the audio stream or file/object and then the video stream or file/object. On activation of the Device, at the start of the day, the Device's application will scan to see if there are any metadata/audio/video sessions that are waiting for upload. The transmission of these files/objects will begin immediately as the Device software is multithreaded in that it can be recording, displaying, queuing and transmitting simultaneously. The Provider will always be shown the number of available minutes they have left in the Device's storage. The Device will always use the direct broadband connection followed by the Wi-Fi connection and if not available, it will then revert to the cellular data connection, if one is available and/or authorized by the Provider. In another embodiment, the Provider can lock down the Device in its secure stand/holder or store it in a cabinet etc. and put the Device in lock down transmission mode through a code where all other Device functions/apps are suppressed (such as email, software 3^{rd} party or system updates etc.) to maximize the resources available to clear all recorded sessions from the Device. In the case of large clinics or institutions, all Devices can be securely networked internally with a much higher internal bandwidth connection to a central secured in-house server that can provide a central store and forward function allowing the individual Devices to be scrubbed and powered down while not in use. Whether the Device is standalone or locally networked, the central cloud server will have the ability to monitor the health of the remote Device and report accordingly as well as provide remote wiping services of both the data and software in the case of a compromised situation. This could utilize location-based services to allow the device to recognize that is being transported away from the site of recording and automatically trigger both a device remote wipe of all existing content which was not uploaded and report the device as stolen through instant messaging to an administrator or authorities.

In another embodiment, the video portion of the session is further broken down into two components, the “snap shot” version and the complete version. The snap shot version takes only a portion of the number of frames per second and either uses this in a streaming or in a batch upload scenario. The rest of the video session is then uploaded when bandwidth becomes available. The two portions of the video session can either be combined at a later state or the snap shot version can be deleted once the complete session is safely deposited in off-line storage. The snap shot version may be comprised of a series of inter frames that are determined by the amount of movement in the scene or it may be set at a fixed time interval. All of the above described techniques are designed to ensure that the Device cannot be “gamed” by the person initiating the recording or to protect against Devices becoming inoperable due to other circumstance before the files/objects can be uploaded.

The Provider at any time can see the status of sessions in the queue and completed. In either case the Provider cannot open or manipulate a session in the queue and all trace of uploaded sessions are removed entirely from the Device once each session is completed real-time streaming or bulk uploaded. Each component of the session will arrive at the secure cloud-based PIPEDA, PHIPA, HIPAA, and HITECH compliant post-processing servers through a different IP path. A folder will have already been created/reserved for this information flow, based on the previously requested case number and will be waiting to receive the various components/objects of the session. Once all components have been received there are numerous automated post processing activities possible. This includes electronically checking the completeness of the files/objects and ensuring that they have not been intercepted and modified. It also includes automatically electronically scanning the metadata, video and audio files/objects without human intervention to check for quality issues and if there is a problem, alerting the specific Provider through various means. Once the complete session files/objects have been accepted by the system, a notification is sent to both parties plus to the outside 3^{rd} party or in-house custodian (Custodian) acknowledging the case number for their reference and how to request access from the custodian of the recorded session in the case of a complaint or investigation. In the case of a post redaction request, it would also be performed at this step in the workflow and the originating file/object would be erased. All access to files/objects at this stage, either manually or electronically are extensively logged in non-erasable files/objects.

The encrypted separate data, audio and video portions of the session folder, stripped of all non-essential metadata, except for the combined case and serial number key, is then sent to a trusted off-site 3^{rd} party HIPAA compliant cloud storage system where it is transferred through an end-to-end direct private connection into deep storage as objects. Such storage may be in the form of tertiary storage that uses robotic tape systems or low cost flash memory. During this operation, the separate objects are stored in three or more different places with the indices stored in three different locations. At this point, no on-line copies of the data/audio/video files/objects are present anywhere in the work flow ranging from the originating Device, to the post processing operation to the trusted 3^{rd} party storage off-line systems. To request a viewing of a specific session or a physical copy, the person or institution must provide a legal written request to the Custodian with specific information to authenticate themselves, to identify the Patient and the Provider, and to provide the date and time of the session in question. The Custodian has access to the base line metadata online (only accessible from their specific IP address) to assist in locating the specific session in question. The Custodian then makes a formal request to the trusted 3^{rd} party off-line storage system to retrieve the specific separate data, audio and video session files/objects. The Custodian then reconstructs the entire session with the separate metadata, audio and video components based on the derived session case/serial key. The Custodian can either provide limited viewing access, with each access logged and/or provide a unique watermarked digital hard copy. Each viewing or hard copy includes a hidden code in the video frames providing digital rights management (DRM) that would point back to the source of an unauthorized transfer or publication of a session as well as provide for authentication of the contents to support digital chain of evidence rules. As all video/audio is stored in a unique CODEC, at this step the Custodian selects the CODEC format that the requester has indicated to facilitate the correct displaying of the requested session on their destination electronic viewing device. The Custodian then converts the reconstructed proprietary CODEC session to a public CODEC to facilitate the authorized viewing of a session. All requests to review recorded sessions results in a notification being sent to both participating parties using their last known contact information.

In another embodiment, the recording Device could actually be provided by the Patient in the form a mobile device, such as smart phone or tablet, where they have downloaded the appropriate app. In this embodiment, all of the work flow steps would remain the same except the business relationship would be with the Patient versus the Provider. In either case the server will always know the status of what is happening on the Patient or Provider Devices relating to recording of sessions. If files/objects are missing or the Device is reported stolen, the server software will remotely lock down the application and erase all encrypted Patient and Provider data.

Various people and organizations need to communicate with the central supplier of the service such as Patients, Providers, Provider boards, associations and colleges, institutions, hospitals, civil lawyers, the judiciary, regulators and the Custodian. In all cases, to protect generic web access from privileged online access, all servers, systems, files/objects and their components are also kept physically and electronically separate.

To support the advancement of health and science through research, as well as to improve the service, all folders are also subjected to an alternative process. This process includes taking the contents of the folder and electronically either removing or redacting all data, audio or video components and images that could identify a specific Patient, Provider, date, time or location. These anonymous secure folders are then subjected to complex data analysis looking for meaningful findings that could be used to further medicine, treatment and drug discovery. Such monetized information could also be used to defray costs of providing the service. The anonymous aggregated folders, stored in a proprietary CODEC, would only be available on-line for processing for short periods of time otherwise they will also be stored off-line in deep storage.

In another embodiment, the role of the central Custodian is enhanced or replaced with the use of a distributed ledger (e.g. a closed Blockchain network) where the base line metadata is stored in various places with appropriate access but the actual content behind the metadata is stored in another location with an additional layer of access protocols. Possible candidates who would host a copy of the distributed ledger, or portions of it, would be the actual Providers, hospitals, boards and associations which manage the Provider's conduct and affairs and the institutions that insure the Providers. To provide access to the recorded sessions, locked away in deep storage, it would require the consent of at least two parties who are distributed ledger Custodians of the metadata.

Those skilled in the art will appreciate that the techniques, logic, and process steps illustrated in the various flow diagrams discussed below, may be altered in a variety of ways to meet specific business cases. For example, the order of the logic steps may be rearranged, sub-steps may be performed in parallel, illustrated logic may be omitted, other logic may be included, etc. One will recognize that some steps may be consolidated into a single step and that actions represented by a single step may be alternatively represented as a collection of sub-steps. The figures are designed to make the disclosed concepts more comprehensible to a human reader. Those skilled in the art will appreciate that actual data structures used to store this information may differ from the figures shown. For example, they may be organized in a different manner; may contain more or less information than shown in these examples; may be compressed and/or encrypted; etc.

### BRIEF DESCRIPTION OF THE DRAWINGS

FIG. 1 is a schematic diagram of the overall work flow of a Patient Provider encounter.

FIG. 2 is a schematic diagram showing the functional components of a device for use in a Patient Provider encounter.

FIG. 3 is a schematic diagram showing software components of a Patient/Provider workflow.

FIG. 4 is a schematic diagram showing functional workflows performable by the device of FIG. 2.

FIG. 5 is a schematic diagram showing data flow of sessions recorded by the device of FIG. 2.

FIG. 6 is a schematic diagram showing storage of content portions of sessions recorded by the device of FIG. 2.

FIG. 7 is a schematic diagram showing third party systems interfacing with a Service Provider.

FIG. 8 is a schematic diagram showing processing of information.

### DETAILED DESCRIPTION

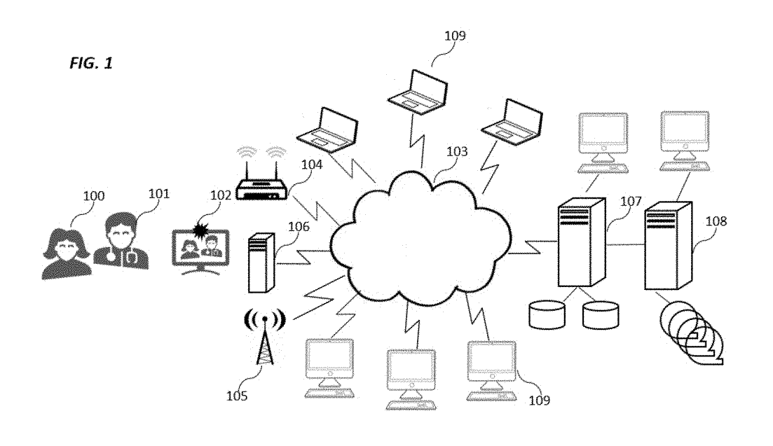

FIG. 1 depicts the overall work flow of a Patient Provider encounter. In this depiction, a Patient 100 enters a service Provider location to meet with Provider 101, enrolls in a video recorded session through various means, such as a mobile tablet Device 102. This Device 102, connects to the cloud 103 by various encrypted communications channels such as Wi-Fi 104, cellular 105 or through a local networked server 106. The cloud is then connected to a central server 107 where various value-added services are provided including handing off the metadata, audio and video components to a secure 3^{rd} party off-line storage Provider 108. Various users 109 will have access to specific services through the cloud for which they are authorized.

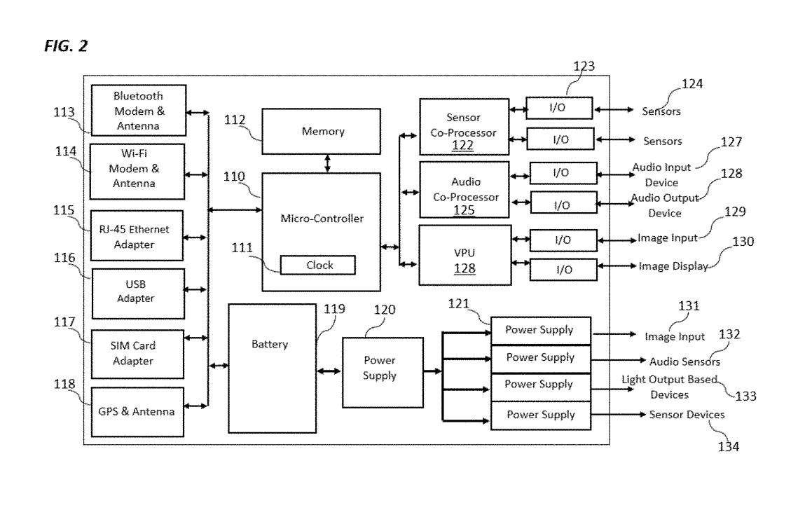

FIG. 2 depicts in more detail what the Device consists of The Device can be a stock, standalone Device such as mobile tablet, separate audio and video external devices connected to a computing device, or a custom built all-inclusive Device. The custom all-inclusive Device would consist of a microcontroller 110, an internal clock 111, memory of various types 112, communications modules such as Bluetooth 113, Wi-Fi 114, Ethernet 115, a USB connection 116, a SIM card adapter 117 and a GPS unit 118. It may also include a battery 119, a master power supply module 120, and specific smaller power supply modules 121. These specific modules 121 would in turn power items such as image capture devices 131, audio devices 132, light output devices 133 and various sensors 134.

To support the input of information there would be various co-processors such as a sensor co-processor 122, an audio co-pro-processor 125, and video processing unit (VPU) 128. These co-processors are in turn attached to various I/O modules 123 to connect to devices such as sensors (e.g. accelerometer, UWB, RTLS devices) 124, audio input devices (e.g. microphone) 127, audio output devices (e.g. speaker) 128, as well as image capture devices and methods (e.g. CCD, IR, LIDAR, photogrammetry, structured light, polarized light, etc.) 129 and image display devices 130. Having specialized processors close to the point of sensor/audio/video capture allows for more advanced features to be provided in real-time as in the case of translation services or avatar creation. In another embodiment, the Device would also include a hardware assist encryption module that may or may not already be part of the microcontroller.

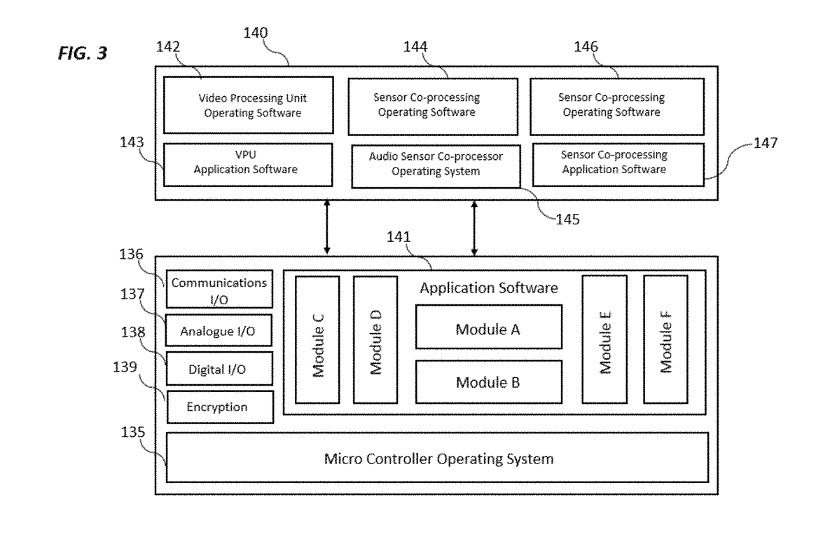

FIG. 3 depicts the type of software components required to meet the proposed secure Patient/Provider recorded workflow. It would include an operating system 135, communications software 136, analogue processing software 137, digital processing software 138, encryption software 139. It would also include software to support the various co-processor 140 such as the VPU operating system 142 and application 143 software, audio co-processing operating 144 and application software 145 and the sensor co-processing operating processing software 146 and application 147. The various user application modules 141 allow for the Device to meet the specific workflow requirements.

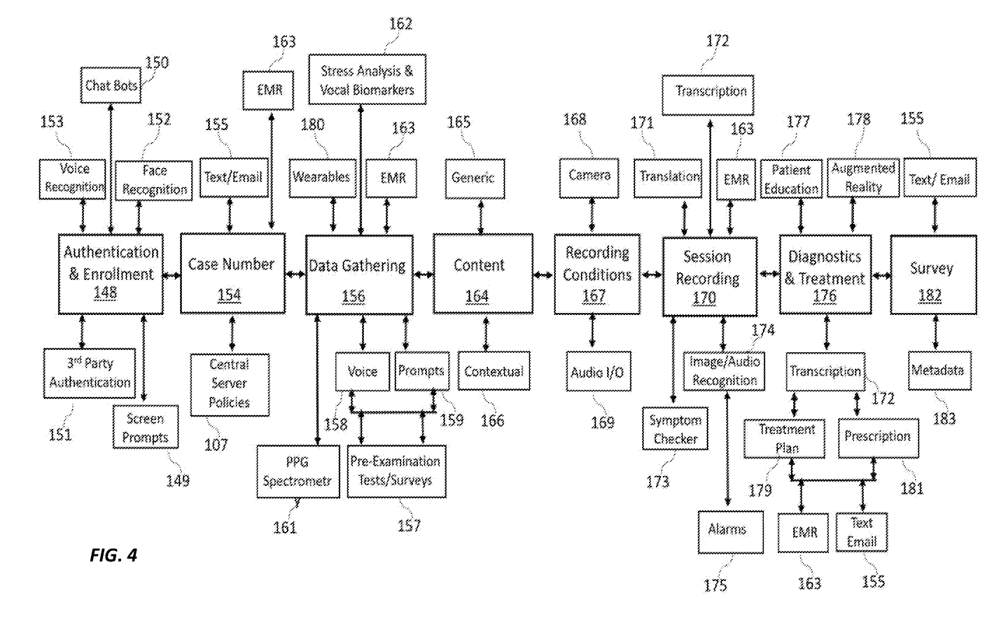

FIG. 4 depicts the various functions and workflow that the Device 102 can perform by using a combination of the operating system components, the co-processors, the system, the network and the custom application software. In this case, the Patient 100 interacts directly with the Device 102 or the system through the web from home or with the administrator at the check-in desk or with the Provider 101 operating the Device directly. The first step is the authentication and enrollment 148 of the Patient which can be done by answering various on-screen 149 or voice prompts (e.g. chatbots) 150 and using image recognition techniques to authenticate the health card against payor or other 3^{rd} party personal authentication sources 151 through API's. In the case of returning Patients, face recognition 152 and voice print recognition 153 can be used to enroll them in a new session. To support enrollment, the Device will request a unique case number 154 from the central server 107 and also assign a sequential serial number to the upcoming session. The Patient 100 at this time is also presented with both the Provider's 101 policies and the required regulatory notices that can be used to create a digital rights contract between the Patient 100 and the Provider 101. The Patient 100 and the Provider 101 can acknowledge the notices and consents through on-screen digital signatures, finger print capture or through a voice print capabilities of the session.

Once the Patient 100 has been authenticated and enrolled, the central server 107 will send secure text/email messages 155 to the Patient or guardian, or have it mailed to their home address, with the case number and how to access the recorded session, in the case of a later formal investigation. It will also log the case number in the Provider's electronic medical record (EMR) system 163.

Regardless of the type of Device 102 used whether it is a stock Device or a custom Device or whether they are controlled through a downloaded application or through a browser app, the use the on-board or connected microphones or cameras are limited to being used for the secure private recording of the pre-agreed session and not for non-approved voyeurism on the part of the Provider 101.

While the Patient 100 is waiting for the Provider 101, the Device 102 can perform data gathering tasks 156 such as running pre-examination tests 157 that the Patient 100 can answer verbally 158 or interact with the Device's screen 159. It can also access and download data from the Patient's wearable devices 180 as well as capture heart rate, heart rate variability, respiration rates and skin composition using techniques such as photoplethysmograthy (PPG), spectrometry, UWB radar, IR or ultrasonic techniques etc. 161. and provide Patient 100 stress and emotional state analysis as well as look for vocal biomarkers 162 using voice and image recognition. Such data captured can be displayed/overlaid on the Devices' screen for the Provider to review, stored in the metadata or even in the electronic medical record (EMR) 163.

While waiting for the Provider to arrive, the Device can display sponsored content 164 that is relevant to the Patient that is either generic 165 or contextual 166 based on Patient or condition specifics.

Once the Provider 101 arrives in the examination room there are various ways to start the formal part of the encounter through manual or automated methods. Manual methods include screen prompts or buttons on the device or through fingerprint recognition. Automated methods include facial/voice recognition, unique vital sign recognition through PPG or the detection of a strong personal electronic/beacon signal. These personal signals can be in the form of pre-registered personal smart phone Wi-Fi hotspot signals or Bluetooth beacon advertising signals. In the case of the smart phone, the signals are pre-registered with the central server but do not need to be paired with the individual Devices in the field. The provider could also be wearing or carrying a personal beacon in the form of a custom low energy Bluetooth or LoRA based beacon or an RFID tag. The recording can start once the advertising signal from the beacon reaches a certain signal strength and/or the Provider can activate a button on the beacon to let the Device know that they are ready to start the recording. All of the above beacon techniques can also be combined with the detection of movement capabilities of the Device. Alternatively, the Provider can use their smart phone to control the Device's recording capabilities and the resulting image can be displayed on the smart phone's display for the benefit of both parties.

In another embodiment, the location of interaction maybe a remote location that is already monitored by other 3^{rd} party sensor systems that are looking at levels of activity in the room and these 3^{rd} party signals could also be used to trigger the system to record.

Once activated for recording, the Device 102 will check the lighting conditions 168 and audio conditions 169 as well as the positioning of the participants in the examination room using computer vision (CV) and AI to provide audio prompts to adjust the environment or participants accordingly 167 before recording starts.

During the start of the examination and the recording segment 170 while the Provider 101 is asking medical and history questions, the Device can be actively listening, transcribing and parsing 172 the responses into a format that can be used to update the electronic medical record (EMR) 163. The Device, using the information obtained as described above can also be recognizing symptoms and through the real-time symptom checker 173, offering possible diagnostic possibilities for the Provider 101 to explore further.

As the Patient 100 and the Provider 101 may not speak the same language, the Device can be actively listening and provide real-time translation 171 to the participants.

Using computer vision (CV) and artificial intelligence (AI), the Device can be monitoring the overall image and audio feeds looking for signs of inappropriate behavior 174 and take appropriate actions at various risk levels such as flagging the session for external review, providing voice prompts/warnings to the participants, calling in the office administrator, and/or dialing 911 etc. 175.

During the diagnostic and treatment recommendation phase 176 of the session, the Provider may call upon medical source material 177 to help explain the medical issue or the treatment options available and/or use augmented reality 178 overlaid onto the Patients affected area to demonstrate what is happening beneath the skin. At this time the Provider 101 can call upon the system to transcribe 172 and parse the recommended treatment plan (audio and video) 179 along with the suggested Patient education support materials into a treatment plan and send the entire package to the Patient 100 by various means 155. Through the above described process, the pharmacist 181 could also be sent the prescription in the form of a text/email or /fax etc. 155 as well as the EMR 163 could be updated. Such information may also be forwarded to a specialist along with other supporting material, such as x-rays, for a referral.

After the Provider 101 has terminated the session, the Patient will be provided with a carefully crafted survey 182 that they can answer through screen or voice prompts either in the examination room, at the front desk or off-site through an online method (e.g. email/text etc.) 155. Survey information can be used to improve the service, highlight areas of satisfaction or dissatisfaction of the encounter or flag areas of concern that the Patient would like the college to investigate. The Patient can be thanked/acknowledge for using the survey by text/email etc. 155. The Provider 101 would also have an opportunity to rank the encounter at the same or later time according to appropriate guidelines from their college, board or association. The survey data at this time may become part of the metadata 183 or application data. The Patient also has the opportunity to withdraw consent at this time of the recorded event so that only the essential metadata of the visit would be stored including their “consent withdrawn” request. Such a withdrawal of consent will trigger a request for confirmation from the provider and a release from both parties that there are no issues with the session.

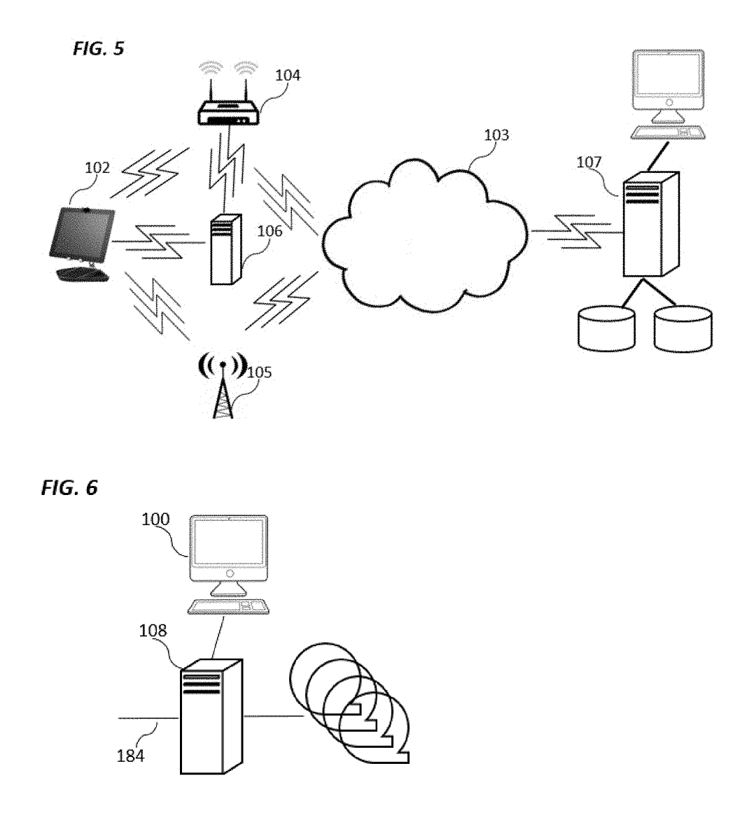

FIG. 5 depicts aspects of the data flow of the recorded sessions as it transits from the recording Device 102 through multiple communications channels to the central cloud based server 107. The central server provides functions such as case number management, temporary folder creation, object creation and file/object encryption management, session store and forward control to off-line services, traffic prioritization, automated quality control of sessions using automated AI techniques, redaction and anonymization services, digital rights management (DRM), sponsored content management, email, FAX and text management and custodian services.

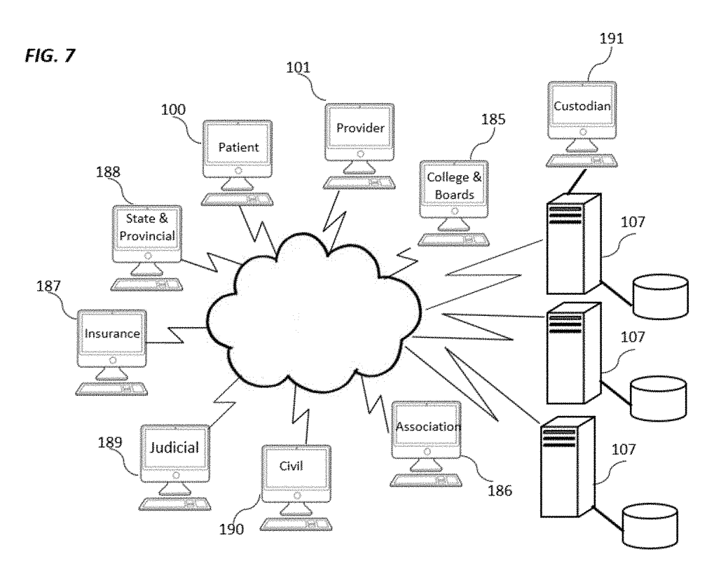

FIG. 6 depicts the methods to store the content portion of sessions in secure offline storage 108. The connection between the central cloud server 107 and the off-line processing server 108 is a privileged one 184 in that the off-line server cannot be contacted through general web access methods. The individual encrypted components of the session, the metadata, audio and video files/objects are individually stored off-line using techniques such as tertiary tape storage or other extremely low cost storage methods.

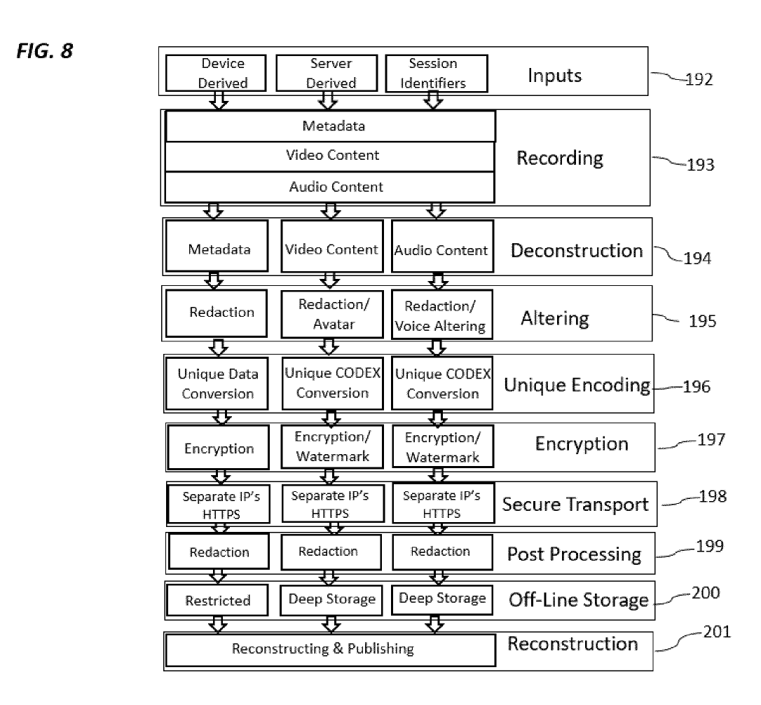

FIG. 7 depicts the various individuals and institutions that will want to access and interface with the service Provider. This includes: The Patient 100 who wants to access general information on the service, pre-signup as a participant to the Provider's service, download their own personal app to present to a non-registered Provider, network with like-minded individuals on the subject at hand and to request an investigation. The Provider 101 who wants to register onto the system. The associated medical college, board 185 or association 186 who needs access to a specific session. The medical malpractice insurance organization 187 who will be representing the Provider 101 who is under investigation. The state or provincial health regulatory organization 188 who needs access to the session in question. The judicial department 187 or criminal lawyers who may be involved with a criminal investigation. The civil lawyers 190 who may be representing Patients 100 and Providers 101 and finally the custodian 191 who makes a final decision on grants of access to the specific session and has the keys to access and distribute the session appropriately.

An alternative embodiment would allow the patient to remotely register, authenticate and request access to the session by using the camera on their computer, laptop or mobile device. The video images collected would be used by the system to perform facial recognition, finger print recognition, retina recognition, and/or heart rate variability through photothermography (PPG).

FIG. 8 depicts the various levels of processing and steps to fragment and encrypt the information collected so that no one failure in the overall system will result in a catastrophic breach of privacy or allow for the gaming of the system. The workflow starts with the inputs 192 generated by the Device, the central server-provided information and those collected by the interaction of the participants of the session. The Device provides metadata, audio and image data as any standard video camera would provide including recording settings (frame speed, aperture, white balance etc.) as well as data/time information read from the Device's microprocessor clock. The server provided data includes a time stamped case number as well as other metadata such as the IP address of the sever supplying the case number and the server's data and time stamp. The session identifiers are generated by either the installed application software or browser based software that collects both system data and variable data that is generated by connected sensors or user generated inputs. System data collected would include the IP address, the MAC address and the cellular IMEI serial number (if cellular communications is used) as well as the sequential serial number of the next video session. Sensor data would include GPS location data and the accelerometer data to indicate that Device has been moved since the last session, as well as ambient light and temperature levels. The attached infrared sensor can be used to sense that there are people in the room and prep the session for a new session or stop the recording of a session if the participants have left the room. The image capture capabilities of the camera can also act as a sensor when combined with computer vision (CV) and artificial intelligence to perform functions such as determining movement in the room, to evaluate back light conditions, to calculate whether the participants are in frame or not and for image recognition of source ID documents and for facial recognition. By combining the camera with PPG technology, the actual HR, HRV and respiration levels of the participants can be recorded. The microphone can also be used as a sensor when combined with automatic speech recognition (ASR) technologies and language translation tools. Additional metadata and application data is collected through application software which both the Patient 100 and the Provider 101 interact with using touch screen prompts and voice commands.

All of the above information is traditionally stored locally, uploaded as a single unit or streamed as a single identifiable recorded unit of information 193. What is proposed is that the metadata, the audio and the video data is deconstructed 194 into at least three or more separate units consisting of the metadata, the audio and the video. In another embodiment, the metadata could be further split in basic metadata to identify a session and all other information, including application data, could be put into an associated application data generated file/object. The separate components or objects could be linked with an encryption key derived from the case number and the serial number of the session.

Either pre- or post-deconstruction, the actual images and voices can be altered in various ways 195 to meet regulatory requirements or privacy requirements. This may include converting the participants into anatomically and dimensionally correct avatars or redacting certain body parts such as faces. Audio voice altering could be employed as well the redaction of personal health information (PHI) from the audio feed.

During the encoding phase 196 the camera records in a specific (CODEC) which can be modified to a custom CODEC which is only known to the custodian. As the data connection to the central server and the offline server is privileged and private, there is no need to display the audio or video files/objects on any known display device in the marketplace. The same is true of most of the metadata that holds Device, session and application derived data so it to can be encrypted in a fashion only known by the custodian as no one else need access to this data. At this phase, all frames of the video are watermarked to assist in supporting a digital chain of evidence to prove that the recording has not been altered after it has passed through the pre-approved altering and redaction phase.

During the encryption phase 197, the three or more separate but linked files/objects can be further encrypted using techniques such as a symmetrical key for the actual content of the message and asymmetrical public key for the message wrapper.

The separate information flows can either be temporarily stored locally on the Device for upload once the session is completed or they can be streamed in real time using secure transport 198. In either case, it is recommended that a static IP address be used at the Device level and that HTTPS be used at the transport level. As mentioned previously, there is the issue of upload bandwidth in various markets and installations. Therefore, all traffic whether it be in a store and forward fashion or a live streaming fashion, will be prioritized in the following scheme: The case number management traffic has priority number one, followed by the metadata then the audio and lastly the video portion of the session. In another embodiment, the video portion of the session is further broken down into two components, the “snap shot” version and the complete version. The snap shot version takes only a portion of the number of frames per second and either uses this in a streaming or in a batch upload scenario. The rest of the video session is then uploaded when bandwidth becomes available. The two portions of the video session can either be combined at a later state or the snap shot version can be deleted once the complete session is safely deposited in off-line storage. The snap shot version may be comprised of a series of inter frames that are determined by the amount of movement in the scene or it may be set at a fixed time interval.

In another embodiment, all three or more of the session component files/objects or steams would follow separate IP paths to the cloud based central server and even rotating ports would be considered if they prove to be of value in further deterring unauthorized access of one or more of the individual components.

Once the information is received at the central cloud based server, it is prepared for post processing 199 and routing to the off-line storage 200. Post processing could include quality assurance services through automated AI routines, pulling out flagged sessions for immediate review, post redaction services, transcription services, survey processing and the making of redacted/altered anonymized and aggregated copies of the files/objects for later big data analysis. Once post processing is completed, the separate metadata, audio and video files/objects are handed off over a privileged and private connection to the secure 3^{rd} party off-line cloud storage supplier. The only thing that is left at the central server is the high-level session information that is only available to the custodian to use to respond to formal requests. Once a particular session is located in this metadata, the provided keys can be used by the Custodian to request and retrieve the separate files/objects and objects of the session from the off-line storage supplier 200.

At the off-line storage stage 200, the individual component files/objects of the session are not stored together as they are stored randomized over various off-storage mediums. Although this technique delays a quick retrieval, it actual meets the business case as retrieval of full sessions requires a long formal process anyways.

At the reconstruction and publishing phase 201, the custodian has received a formal, authenticated and legal request for a specific session. In another embodiment, the requesting patient would be required to submit biometric evidence of identity to link their fingerprint, retina scan, facial features, electrocardiogram tracing or other biometric information to the original session in question to trigger the process of reconstruction. The custodian would use the limited local session data to locate the session in question and through their private and privileged connection to the 3^{rd} party off-line storage supplier, they would make the formal request. The 3^{rd} party off-line storage supplier would then authenticate the request and locate all of the components or objects and forward them as they become available over the privileged point-to-point connection. The custodian would then assemble the various components into a single file and review them using the private custom CODEC. The custodian would then convert the re-constructed session file into a common CODEC that would allow the requester to view the session on a zero-footprint basis on their display device of choice or a digital copy would be made. Regardless of the type of sharing, each formal requester of the session would get a different digitally watermarked version so that if there is a downstream breach of privacy it could be traced back to the source.

A number of embodiments have been described where it is understood that various modifications may be made without departing from the spirit and scope of the invention.

What is claimed is:

1. A system for securely recording, uploading and managing data, audio and video relating to sensitive private and privileged person-to-person encounters, comprising:
a secure recording device for collecting personal data, metadata, audio and video information of a user(s);
a secure communication system to transfer said sensitive and confidential data; and
a secure server for processing, organizing, storing and disseminating said sensitive and confidential data.

2. The system of claim 1, in which the input from the recorded session, comprised of application data, video metadata, audio files and video are dynamically converted into separately linked uniquely encoded objects whose content, format, encoding and linkage algorithm is only known by the receiving server.

3. The system of claim 1, where the method of transmitting the linked but separate objects are packed up for secure transmission using an asymmetrical encryption key for the message wrapper and symmetrical encryption key for the message contents.

1. The system of claim 1 where the separately encoded and encrypted but linked objects are transmitted through separate secure communications channels using SSL at random and at different times to different IP addresses to reach the final end servers.

5. The system of claim 1 where the separately encoded and encrypted but linked objects can be prioritized for transmission based on their object data size so that application data, metadata and audio content will have transmission priority over larger video based content objects.

6. The system of claim 1, where the separately encoded and encrypted but linked objects can be prioritized not only based on object size for bulk uploading but also for streaming in real time where lighter data object streams will get priority over heaver video content object streams.

7. The system of claim 1, where the separate but linked video related objects will be further subdivided into a much smaller snap-shot version, to be used in bulk uploading or in live streaming, comprised of either video frames based on a defined timed interval or based on inter frames which will increase their priority in the transmission algorithm.

8. The system of claim 1, where the separately encoded and encrypted but linked objects are stored off-line in a randomized fashion that can only be retrieved and reconstructed by a custodian who has privileged and logged access to the keys to provide digital evidence in the case of a formal investigation request.
